(19) United States
(12) Patent Application Publication
(10) Pub. No.: US 2016/0320400 A1
(43) Pub. Date: Nov. 3, 2016

(54) BIOSENSORS

(71) Applicant: Protein Technologies Limited, Manchester, Greater Manchester (GB)

(72) Inventors: Farid Khan, Manchester (GB); Leopoldo Napoleón Herrera Rodriguez, Manchester (GB); Tariq Ali, Manchester (GB)

(73) Assignee: Protein Technologies Limited, Manchester, Greater Manchester (GB)

(21) Appl. No.: 14/391,946

(22) PCT Filed: Apr. 11, 2013
(86) PCT No.: PCT/GB2013/050940

§ 371 Date: Oct. 10, 2014

(30) Foreign Application Priority Data
Apr. 11, 2012 (GB) 1206368.1

(51) Int. Cl. G01N 33/68; C12N 15/62

(52) U.S. Cl. CPC G01N 33/68; C12N 15/625; C07K 2319/21; C07K 2319/60; C07K 2319/50; C07K 2319/20

(57) ABSTRACT

The invention relates to biosensors, more particularly to protein biosensors and their use to indicate the formation of denaturing ice crystals in samples. The invention includes the use of a protein as a biosensor to indicate denaturing ice-crystal formation in a sample, wherein the protein is denaturable by denaturing ice crystals from a non-denatured state to a denatured state and wherein said non-denatured and denatured states are associated with detectably different indications, e.g. colour, fluorescence, bioluminescence, protein activity, etc. Biosensor compositions according to the invention preferably comprise a protein comprising a ligand-binding domain and a ligand bindable by the ligand-binding domain. Particularly good results are obtained using proteins, especially fluorescent proteins, having at least one polyhistidine tag and a ligand comprising imidazole or an equivalent polyhistidine-binding component. For example, polyhistidine-tagged Ds Red Express2 (Ds Red2) is especially preferred for use in the invention. On denaturation by denaturing ice crystals, this protein undergoes a marked change of colour indication from red to colourless. The invention is suitable for indicating the formation of denaturing ice crystals in a range of different samples susceptible to ice crystal damage, e.g. foodstuffs, pharmaceutical preparations, laboratory reagents, cells, tissues, etc.

The present invention relates to biosensors, more particularly to the use of protein biosensors to indicate the formation of denaturing ice crystals in samples.

Proteins, cells and whole tissues can be preserved by freezing to sub-zero temperatures; at such temperatures, all biological activity effectively stops. However, freezing can also damage proteins, cells and whole tissues due to the undesirable formation of ice crystals which can cause the mechanical disruption of cellular membranes and the physical shearing and denaturation of individual proteins and proteinaceous compounds.

Freezing takes place in response to thermodynamic and kinetic factors, either of which may predominate at a particular stage in the freezing process. Major thermal events are accompanied by a reduction in the heat content of material during the freezing process. The material first cools down to the temperature at which freezing may occur, but, before ice can actually form, a nucleus or a seed is required upon which the crystal can grow; the process of producing this seed is termed nucleation. Once the first crystal appears in the solution, a phase change occurs from liquid to solid with further crystal growth. Therefore, nucleation serves as the initial process of freezing, and can be considered as the critical step that results in a complete phase change.

The freezing point is defined as the temperature at which the first ice crystal appears and the liquid at that temperature is in equilibrium with the solid. If the freezing point of pure water is considered, this temperature will correspond to 0° C. (273° K). However, the process becomes more complex in freezing of materials containing both free and bound water. Bound water does not freeze even at very low temperatures. Unfreezable water contains soluble solids, which cause a decrease in the freezing point of water lower than 0° C. During the freezing process, the concentration of soluble solids increases in the unfrozen water, resulting in a variation in freezing temperature. Therefore, the temperature at which the first ice crystal appears is commonly regarded as the initial freezing temperature.

Freezing time and freezing rate are the most important parameters in designing freezing systems. The quality of a frozen product (for example, a food product or a biopharmaceutical) is mostly affected by the rate of freezing, with time of freezing being calculated according to the rate of freezing. For industrial applications, these are the essential parameters in the process when comparing different types of freezing systems and equipment.

Generally, rapid freezing results in better quality frozen products in comparison to slow freezing. If freezing is instantaneous, there will be more locations within the product for crystallization to occur. In contrast, if freezing is slow, the crystal growth will be slower with few nucleation sites resulting in larger ice crystals. Large ice crystals are known to cause both mechanical damage to cell walls and cell dehydration. Thus, the rate of freezing of materials such as food and biopharmaceutical products is extremely important due to the effect that it has on the size of ice crystals, cell dehydration, and cell wall damage.

Ice-crystal damage in materials such as biological samples is particularly observed upon slow freezing where large ice crystals are formed and a low ice-liquid area interface is created that damages cells and causes proteins to aggregate. Slow freezing typically occurs with commercial and domestic freezers that freeze food and liquids to −20° C.; most household and laboratory freezers maintain temperatures of approximately −23° C. to −18° C. (−10° F. to 0° F.). Fast freezing or flash freezing form smaller crystals because a relatively large ice-liquid area interface is created, thereby allowing conditions that are desirable for the cryopreservation of proteins. Flash freezing may be achieved, for example, by immersion of samples in liquid nitrogen, exposure to solid carbon dioxide or in ultra-low temperature freezers such as −80° C. freezers.

There are many methods and practices in the food and life science industries for preventing or minimizing the damage caused by the formation of ice crystals. These range from the simple labelling of finished, packaged products (which must be refrigerated as they pass through the cold chain supply network but must not be frozen), through to complex formulations and cryopreservation techniques for pharmaceutical preparations (which must necessarily be preserved or stored at sub-zero temperatures during or after manufacture but must be thawed prior to administration). With respect to the former, incidents of accidental freezing are quite common and represent both an economic burden upon manufacturers and a not insubstantial risk to public health.

With a view to the protection of public safety, labels indicating whether a given product has been frozen have been marketed for several years. None of these labels, however, are true ice crystal sensors—nor indeed do they purport to be such. Rather, they are simply designed to register the freezing event per se as opposed to the formation of ice crystals—a situation which may or may not come about depending on a number of factors including the presence of nucleating particles, rates of temperature decrease versus time, degree of hydration and the presence or absence of buffers and cryopreservatives. Such freeze labels are either based on the dispersion of solid particles in liquid media or membranes which fracture on freezing and release a stain. See, for example, U.S. Pat. No. 7,343,872 ‘Freeze Indicators Suitable for Mass Production’ and US 2010/0162941 ‘Freeze Indicators, Components therefor and Preparative Processes.’

More sophisticated devices are also marketed incorporating electronic temperature sensors. For example, the ‘ibug’ (ibug Sensors Ltd, Saffron Walden, UK) monitors the temperature conditions to which products are subjected during transport. On receipt of the product, the recipient depresses the tube cap and a red or green LED will illuminate indicating whether the pre-set temperature limits have been exceeded. Although an efficient recorder of temperature variation, this device does not detect the formation of ice crystals and therefore cannot be used to determine whether cellular membranes or proteins have been damaged by such. With all of the above solutions, a given product may only have been exposed to freezing temperatures for a few seconds—sufficient to cause the label or temperature indicator to register a freezing event but insufficient for ice crystals to have formed and therefore for the product to have been adversely affected by them.

An additional problem with both labels and electronic temperature sensors is cost. Pharmaceutical preparations can be damaged by heat and, as a result, vaccine vials have long been individually labelled with blister pack stickers which change colour above a certain temperature. According to PATH—an international non-profit healthcare charity—the cost of such stickers adds approximately $0.01 to each vial. The production costs of freeze labels and electronic temperature sensors are considerably higher, however, because the devices themselves are more elaborate and require more complex fabrication. They are also physically larger. For example, the ‘Freeze Watch’™ label (3M Corporation, US) is 3 cm long. As a result, although vaccine vials are routinely individually labelled with heat sensors they are not individually labelled with freeze sensors. Instead, a single freeze sensor is positioned somewhere inside or on the outside of a packaged batch of vaccines—a suboptimal solution as temperatures may vary considerably from vial to vial throughout the package as it travels along the length of the cold chain supply network.

Turning now to pharmaceutical preparations which must necessarily be preserved or stored at sub-zero temperatures during or after manufacture but must be thawed prior to administration, freezing and cryopreservation technologies have been developed over many years. However, particularly in newly emerging therapies such as stem cells, these technologies can still be somewhat unreliable, with few standardized protocols, widespread wastage, spoilage and sub-optimal yields.

The cryopreservatives that are currently added to inhibit the formation of intra- and extra-cellular ice crystals but which are associated with serious problems of toxicity and immunogenic response include dimethyl sulfoxide (DMSO) and bovine serum albumin. At present, many laboratories and research institutes are engaged in attempting to reduce the amounts of these compounds or to replace them altogether with other more benign cryoprotectants. What these researchers currently lack is not just a freeze sensor that they may use during storage and transportation, but moreover a laboratory reagent, which they may employ to measure the formation of ice crystals in their experimental investigations.

The present invention has been made from a consideration of the aforementioned problems.

The present invention provides for the first time a protein biosensor for indicating the formation of damaging ice crystals.

According to a first aspect of the present invention, there is provided the use of a protein as a biosensor to indicate denaturing ice-crystal formation in a sample, wherein the protein is denaturable by denaturing ice crystals from a non-denatured state to a denatured state and wherein said non-denatured and denatured states are associated with detectably different indications.

Thus, the denaturation of the protein by denaturing ice crystals, i.e. the change from the non-denatured, native state to the denatured state, can be detected through the change in the associated indication. By storing the protein biosensor under the same conditions as the sample, therefore, it is possible to determine whether denaturing ice crystals have formed in the sample.

If there has been no freezing of the sample and the protein, and hence no ice-crystal formation, the protein remains in its native, non-denatured state and there is no change in the associated indication. Further, if there has been freezing of the sample and the protein under conditions such that the formation of ice crystals capable of denaturing the protein is avoided, the protein remains in its native, non-denatured state and there is no change in the associated indication. On the other hand, if the sample and the protein are frozen under conditions in which denaturing ice crystals have formed, the protein will be denatured by the ice crystals from the non-denatured state to the denatured state and this will be detectable through the change in the associated indication. From this change, it can be deduced that denaturing ice crystals have also formed in the sample.

Thus, the present invention is suitable for indicating the formation of denaturing ice crystals in a range of different samples susceptible to ice crystal damage. Given that it is essentially the damage done to constituent proteins by ice-crystal formation that spoils or renders products such as foodstuffs, pharmaceutical preparations, laboratory reagents, cells, tissues, etc. unusable, the protein biosensor as herein described provides an ideal means for detecting whether denaturing ice-crystal formation has occurred resulting in damage to the structural integrity of the proteins, cells or tissues of which the samples are composed.

According to a further aspect of the present invention there is provided a biosensor composition for indicating denaturing ice-crystal formation in a sample, the composition comprising a protein, wherein the protein is denaturable by denaturing ice crystals from a non-denatured state to a denatured state and wherein said non-denatured and denatured states are associated with detectably different indications.

In use, the biosensor composition requires water in order to operate in the detection of denaturing ice crystal formation. Thus, water may be provided as a further component of the biosensor composition. Alternatively, the water may be provided separately for use of the biosensor composition.

Typically, the water as referred to herein is in the form of an aqueous solution, i.e. a solution in which one or more solutes are dissolved in water. The aqueous solution may help to maintain the protein in its non-denatured state.

According to a further aspect of the present invention there is provided a method of indicating denaturing ice-crystal formation in a sample stored under storage conditions, the method comprising:

(i) storing under said storage conditions a biosensor composition comprising a protein, wherein the protein is denaturable by denaturing ice crystals from a non-denatured state to a denatured state and wherein said non-denatured and denatured states are associated with detectably different indications, and water; and

(ii) detecting any change of indication.

According to a further aspect of the present invention there is provided a protein for use in a biosensor composition for indicating denaturing ice-crystal formation in a sample, wherein the protein is denaturable by denaturing ice crystals from a non-denatured state to a denatured state and wherein said non-denatured and denatured states are associated with detectably different indications.

It will be appreciated that the present invention is applicable to a wide range of different samples and may be employed in a variety of different fields. The present invention is particularly useful in the storage and transport of samples that are susceptible to denaturation by ice crystals. The sample may comprise biological materials, such as human-derived products. By way of example, the sample may comprise materials such as pharmaceuticals, antibodies, enzymes, biopharmaceuticals, proteinaceous laboratory reagents, tissues, cells and cell research products, including stem cells, and blood and blood products. The present invention may be useful, for example, in a biobank for storage of tissue and cells.

The sample may be, for example, a food product. For example, flash freezing (or blast freezing) is used in the food industry to quickly freeze perishable food items. In this case, food items are subjected to temperatures well below the melting/freezing point of water (32° F. or 0° C.), causing the water inside the foods to freeze in a very short period without forming large crystals, thus avoiding damage to cell membranes. Flash freezing techniques are also used to freeze biological samples fast enough that large ice crystals cannot form and damage the sample. This rapid freezing is typically carried out by submerging the sample in liquid nitrogen or a mixture of dry ice and ethanol.

The sample may comprise a proteinaceous material.

The present invention is also particularly useful in relation to products and processes that require slow freezing. An example is in the manufacture of ice cream. The body of ice cream is referred to as the ‘whole mass’, the firmness, resistance and texture of which is due to the production of fine ice-crystals in the ice cream. The size of the ice-cream crystals formed during freezing depends on two manufacturing processes; upon the rate of the freezing process and the rate of turning (stirring) in the freezer. The present invention provides for inexpensive monitoring of the first process, i.e. the slow rate of freezing in which results in large ice-crystal formation—a condition which is essential for the manufacturing and processing of ice cream.

It will be appreciated that the storage conditions are typically cold storage conditions, the present invention being particularly suited for use in relation to temperature-sensitive samples requiring cold storage. The present invention provides an indication of denaturing ice-crystal formation in a sample stored under any storage conditions in which such ice crystals may form. As noted above, large, denaturing ice crystals are typically formed by relatively slow freezing at higher (i.e. less cold) temperatures. Domestic freezers may provide a typical freezing rate of <5° C./min and typically operate at temperatures of −20° C. or higher. Under these conditions, denaturing ice crystals are typically formed and denaturation of the biosensor protein will result. On the other hand, rapid freezing under colder temperatures, such as flash freezing at temperatures of −80° C. tends to form smaller, non-denaturing, crystals because a relatively large ice-liquid area interface is created. Under these conditions, denaturing ice crystals tend not to form, and the protein biosensor therefore remains in its non-denatured state.

In particularly preferred embodiments of the present invention, the protein is at least partially irreversibly denaturable by denaturing ice crystals. In other words, the denaturation of the protein from the non-denatured state to the denatured state caused by denaturing ice crystals results in some irreversible change in the protein structure. In turn, this results in an irreversible detectable change in the associated indication. This is especially advantageous, as it provides for a lasting indication that the protein, and hence the sample, has been exposed to storage conditions in which denaturing ice crystals were formed, even if the storage conditions subsequently change to conditions in which such damaging ice crystals would not result. In this way the biosensor provides a tamper-proof indicator of spoilage in which ice-crystal damage of the sample has occurred. Thus, an undesirable freezing event during storage of the sample causes an irreversible change to the indication associated with the denatured state of the protein, even if subsequently the sample is correctly frozen.

The denaturation of the protein by denaturing ice crystals to the denatured state preferably results in a change of associated indication that is detectable when the protein, or the biosensor composition containing the protein, is either frozen or thawed. This allows for the indication of protein denaturation to be detected in situ, where the protein or biosensor composition containing the protein, and typically also the sample, is in a freezer. Usefully, this permits the ready identification and discrimination of ice-damaged frozen samples from undamaged (i.e. correctly stored) samples. This is particularly useful where a large number of samples are to be stored, for example in a biobank which may retain many thousands of stored cells. This also negates the need for the time-consuming thawing of samples before inspection or testing for function/viability.

The protein should be subjected to the same conditions as the sample, thereby ensuring an accurate indication of any denaturing ice-crystal formation. The protein, or the biosensor composition containing the protein, may be introduced into or otherwise combined with the sample itself, for instance prior to storage or transportation of the sample. However, this may not be possible or desirable in all circumstances. Alternatively, therefore, the protein, or the biosensor composition containing the protein, may be stored separately but in conjunction with the sample. By way of example, the protein, or the biosensor composition containing the protein, may be stored separately in or on containment means containing the sample, such as packaging means.

In this regard, the protein may be incorporated into a material or composition applied to samples or their packaging, for example by printing, encapsulation or a similar process. Encapsulation ensures that the biosensor protein is maintained in an aqueous environment during use. The biosensor protein may be applied as part of a bar code or any other identification means, thereby facilitating identification of samples and whether they have been safely frozen.

An ‘indication’ may be anything that serves to indicate to a user the associated state (i.e. non-denatured or denatured) of the protein, such as, for example, colour, fluorescence, bioluminescence, protein activity, etc. The indication may, for example, be visually detectable and/or detectable using instrumentation such as a spectrophotometer, for example an absorbance spectrophotometer or fluorescence spectrometer, or a luminometer.

Either or both of the detectably different indications associated with each of the non-denatured and denatured states of the protein may result directly from a change in the protein between the non-denatured and the denatured state. In other words, the change between the non-denatured and the denatured state may bring about a detectable change in the protein itself, such as a detectable structural change.

Additionally or alternatively, said detectably different indications although associated with the non-denatured and denatured states may arise from an indirect effect that a change in the protein has on another substance, a change in that other substance being detectable.

The protein may be naturally sensitive to denaturing ice crystals.

The protein may be modified to introduce or improve susceptibility to denaturation by denaturing ice crystals.

In some embodiments, an accessory component may be used in order to introduce or improve susceptibility to denaturation by denaturing ice crystals. For instance, the accessory component may interact with the protein to induce a particular structural conformation or arrangement that is detectably disrupted on denaturation by denaturing ice crystals. Additionally or alternatively, the accessory component may interact with the protein to bring about binding or association between individual protein molecules that are detectably disrupted on denaturation by denaturing ice crystals.

The protein may be modified in order to introduce or improve binding of such an accessory component. For instance, the accessory component may comprise a ligand and the protein may be provided with at least one corresponding ligand-binding domain. Examples of suitable pairs of ligands and ligand-binding domains include but are not limited to imidazole and polyhistidine; glutathione and glutathione S-transferase (GST); biotin and streptavidin; and luciferin and luciferase. It will be understood that functional analogues and derivatives of any ligand may also be used. Similarly, functional variants, fragments and derivatives of the ligand-binding domains are encompassed.

The protein may be a modified protein having at least one ligand-binding domain comprising a polyhistidine tag and may be used in conjunction with a ligand bound by a polyhistidine tag. The polyhistidine tag may, for example, have at least five histidine residues and typically has six histidine residues, although longer polyhistidine tags may also be used. Modification of proteins with polyhistidine tags is known to those skilled in the art (Khan et al. (2006) Analytical Chemistry 1; 78(9):3072-9), although the present application describes for the first time the use of polyhistidine-tagged proteins as biosensors to indicate denaturing ice-crystal formation in a sample.

The ligand bound by a polyhistidine tag may comprise, for example, imidazole and its derivatives and analogues, or other compounds comprising aromatic groups or pyrrolic functional groups. These include amino acids, such as histidine, histidine-containing peptides, porphyrin structures and aptamers that specifically bind polyhistidine tags. By way of example, the concentration of imidazole or similar ligands may be in the range of between 10-500 mM, such as 25-400 mM, 50-300 mM, 75-250 mM, 100-200 mM or 125-175 mM. Good results have been observed at around 150 mM.

Protein concentrations may vary but are typically between 0.1-5 mg/ml, such as 0.25-4 mg/ml, 0.5-3 mg/ml, 0.75-2 mg/ml or 1-1.5 mg/ml. Good results have been observed at around 1 mg/ml.

The inventors have observed particularly good results using proteins, especially fluorescent proteins (FPs), having at least one polyhistidine tag and a ligand comprising imidazole or an equivalent polyhistidine-binding component.

According to a further aspect of the present invention there is provided a biosensor composition comprising:

a protein, wherein the protein is denaturable by denaturing ice crystals from a non-denatured state to a denatured state and wherein said non-denatured and denatured states are associated with detectably different indications, and wherein the protein comprises a ligand-binding domain; and

a ligand bindable by the ligand-binding domain.

In use, the biosensor composition requires water in order to operate in the detection of denaturing ice crystal formation. Thus, water may be provided as a further component of the biosensor composition. Alternatively, the water may be provided separately for use of the biosensor composition.

Without wishing to be bound by any particular theory, the inventors have hypothesised that the imidazole (or imidazole analogue or similar polyhistidine tag-binding accessory component) effects intermolecular binding with the polyhistidine-tagged proteins, thereby rendering them particularly susceptible to the physical forces involved in the growth of large, denaturing, ice crystals and resulting in their unfolding. This process results in denaturation, followed by aggregation with concomitant, and preferably irreversible, change of associated indication. Where FPs are used, this will generally involve loss of both colour and fluorescence. Under conditions in which there is no significant formation of denaturing ice crystals, such as flash freezing at −80° C., the indication associated with the non-denatured state of the protein remains unchanged. Thus, where FPs are used, both the fluorescence and colour of the FPs are generally unaffected, even after many cycles of being correctly frozen and thawed.

Proteins may be modified by the provision of a polyhistidine tag at the N-terminal and/or the C-terminal and/or at any other suitable part of the protein. The use of more than one polyhistidine tag may result in improved sensitivity of the modified protein to denaturation by denaturing ice crystals and faster response times in terms of the indication of denaturation by denaturing ice crystals.

The protein, and any accessory component(s), may be used with other components, such as buffers, salts, regulators, preservatives, etc., which may form part of the biosensor composition. These additional components may help to maintain the protein in its non-denatured state. Any suitable components may be used. By way of example, a buffer may comprise phosphate buffered saline (PBS). Other examples include, but are not limited to, any one or more of: nitrilotriacetic acid (NTA), NTA derivatives, 3-{[tris(hydroxylmethyl)methyl]amino}propanesulfonic acid, N,N-bis-(2-hydroxyethyl)glycine, tris(hydroxymethyl)methylamine, N-tris-(hydroxyl-methyl)methylglycine, 4-2-hydroxyethyl-1-piperazineethanesulfonic acid, 3-(N-morpholino)propanesulfonic acid, piperazine-N,N′-bis(2-ethanesulfonic acid), saline sodium citrate, 2-(N-morpholino)ethanesulfonic acid, acetic acid, sodium acetate, K₂HPO₄, KH₂PO₄, KCl and EDTA.

In embodiments of the present invention, the non-denatured and denatured states of the protein are associated with detectably different electromagnetic radiation indications. Thus, any change between the non-denatured and denatured state is associated with a corresponding change in electromagnetic radiation, such as a change in absorbance or emission, including fluorescence emission and luminescence.

A material's absorption spectrum is the fraction of incident radiation absorbed by the material over a range of frequencies. The absorption spectrum is primarily determined by the atomic and molecular composition of the material. Radiation is more likely to be absorbed at frequencies that match the energy difference between two quantum mechanical states of the molecules. The absorption that occurs due to a transition between two states is referred to as an absorption line and a spectrum is typically composed of many lines. The frequencies where absorption lines occur, as well as their relative intensities, primarily depend on the electronic and molecular structure of the molecule. The frequencies will also depend on the interactions between molecules in the sample, the crystal structure in solids, and on several environmental factors (e.g., temperature, pressure, electromagnetic field). The lines will also have a width and shape that are primarily determined by the spectral density or the density of states of the system.

‘Absorbance’ refers to a measure of the capacity of a substance to absorb light of a specified wavelength, which is equal to the logarithm of the reciprocal of the transmittance.

‘Fluorescence’ refers to the emission of light by a substance that has absorbed light.

‘Luminescence’ refers to the emission of light by a substance not resulting from heat; it is thus a form of cold body radiation. It can be caused by chemical and biochemical reactions (bioluminescence), electrical energy, subatomic motions, or stress on a crystal.

In preferred embodiments of the present invention, the non-denatured and denatured states of the protein are associated with detectably different visual indications. Thus, any change between the non-denatured and the denatured state is associated with a corresponding visual change, which provides for ready detection.

The visual indications typically correspond to changes in electromagnetic radiation in the visible range of wavelengths from 390 nm to 790 nm. In most cases, emitted light has a longer wavelength, and therefore lower energy, than absorbed radiation. The visible spectrum is the portion of the electromagnetic spectrum that is visible to (i.e. detectable by) the human eye. Electromagnetic radiation in this range of wavelengths is also commonly referred to visible light or as simply ‘light’.

The visual indications may correspond to visual fluorescence indications, being the emission of light of wavelengths from 390 nm to 790 nm. A typical human eye will respond to wavelengths from about 390 to 750 nm. In terms of frequency, this corresponds to a band in the vicinity of 400-790 THz.

In preferred embodiments of the present invention, the non-denatured and denatured states of the protein are associated with detectably different colour indications. Thus, any change between the non-denatured and denatured state is associated with a corresponding colour change within the visible spectrum.

The colour change may, for instance comprise a change from a first colour indication to a detectably different second colour indication; or it may comprise a detectable partial or substantially complete decrease in colour; or it may comprise a detectable increase in colour, including a detectable appearance of a colour indication where previously colour was essentially absent.

Electromagnetic radiation is characterized by its wavelength (or frequency) and its intensity. The visible spectrum corresponds to wavelengths between 390 nm to 750 nm. The colour indications may comprise absorbance in the red (635-700 nm), orange (590-635 nm), yellow (560-590 nm), green (490-560 nm), blue (450-490 nm) or violet (400-450 nm) regions of electromagnetic radiation. Similarly, fluorescence emission (which results from excitation at lower wavelength that the emission wavelength) may comprise red (635-700 nm), orange (590-635 nm), yellow (560-590 nm), green (490-560 nm), blue (450-490 nm) or violet (400-450 nm).

The ‘protein’ as used herein may comprise a full-length protein or polypeptide, or any functional component, variant, derivative or fragment thereof. A full-length protein contains the complete structure of a transcribed gene which may consist of single or multiple domains of discrete secondary structure. A polypeptide is defined as two or more amino acids that are linked together to form peptidic bonds and form part of the primary sequence of the protein. Functional components are secondary structural elements which are involved in the functional regions of the protein, such as binding sites.

The protein may comprise a natural protein or an engineered protein.

Protein engineering is a set of procedures by which protein structure and function are changed or created in vitro by altering existing genes or synthesising new structural genes that direct the synthesis of proteins with sought-after properties. To achieve these properties a number of techniques are used, such as molecular modelling, site-directed mutagenesis and directed molecular evolution. The resultant engineered proteins may consist of proteins with primary sequences and structures that are very different to the corresponding original wild-type sequence and structure.

The protein used in the present invention may be an engineered protein engineered to improve its properties including fluorescence excitation and/or emission, stability, and sensitivity to ice crystal damage and/or to introduce functional domains such as metal binding sites.

Embodiments of the present invention where the non-denatured and denatured states of the protein are associated with detectably different colour indications typically employ a coloured or fluorescent protein. Such proteins have tertiary structures containing active chromophores or fluorophores, which become unstructured and inactivated on ice-crystal denaturation.

The protein may be associated with other peptide- or non-peptide groups. Non-peptide groups include prosthetic groups or cofactors. The non-peptide groups may provide the protein with colour or fluorescence.

The protein may comprise a naturally coloured or naturally fluorescent protein. The protein may comprise a fluorescent protein (FP) with a beta-barrel structure, such as a green fluorescent protein (GFP). There are a number of known fluorescent proteins that essentially span the visible wavelength. Alternatively, the protein may comprise a naturally coloured or naturally fluorescent protein without GFP-like beta-barrel structure. Non-limiting examples include metalloproteins, such as haemoglobin, plastocyanins, rubredoxins, cytochromes, and chlorophyll-containing proteins.

GFP is a protein which fluoresces bright green on excitation with UV or blue light. The gene for GFP was originally isolated from the jellyfish Aequorea victoria. Many GFP-like proteins have been identified. These proteins have a range of colours and fluorescence characteristics and are collectively known as fluorescent proteins (FPs). The unique properties of the fluorescence/colouration of FPs are due to the autocatalytic formation of their chromophores, which are usually buried inside the protein structure. This family of FPs is also known as genetically-encoded fluorescent proteins, as the proteins can spontaneously become coloured or fluorescent without the need of co-factors and can be recombinantly expressed in a range of expression systems.

FPs make excellent biosensors by virtue of their high fluorescence, and are preferred for use in certain embodiments of the present invention. When fluorescent proteins are denatured (i.e. where a complete or partial loss in structure occurs) there is concomitant loss in absorbance and fluorescence due to the structural displacement of the protein structure or chromophore environment.

The sensitivity of the visual indication may be improved using ultraviolet (UV) light, for instance with a UV lamp, such as a portable UV lamp of the kind commonly employed for the detection of counterfeit currency. UV light is electromagnetic radiation with a wavelength shorter than that of visible light, in the range 100 nm to 400 nm, and is not detected by the human eye. UV lamps typically emit light at wavelengths between 200-400 nm. Most proteins comprise aromatic residues such as tyrosine, tryptophan and phenylalanine, thereby rendering them UV active. Typically proteins can be excited by UV light at 260-290 nm and emit fluorescence at 300-330 nm. However in the case of FPs, the chromophores and surrounding aromatic residues may be excited in the UV region (i.e. 260-300 nm, but typically at 280 nm) and partake in fluorescence energy transfer to the chromophore thereby allowing emission of light at longer wavelengths (also known as ‘Stoke's shift’). In comparison to absorbance, fluorescence as a method of optical detection is generally at least an order of magnitude more sensitive. Therefore, in the present invention, the sensitivity of detection by the eye of the change in associated visual indication on denaturation of the biosensor protein may be enhanced using UV excitation and the intensity of the light emitted by fluorescence can be determined visually. Usefully, this allows the protein biosensor to operate at lower concentrations.

FPs can be expressed recombinantly in a host of vectors including bacteria such as E. coli, mammals, plants, yeast and fungi, using techniques well known to those skilled in the art.

FPs such as GFP are known to have monomeric, tightly folded beta-barrel structures and exhibit fluorescence when the chromophore is buried in its native folded state. It is known that on denaturation by heat, pH or chemicals, GFP unfolds, the chromophore is displaced and there is a loss in the characteristic green fluorescence and subsequent loss in green colour. Many optimised GFP variants have been reported, including enhanced GFP (EGFP) which has mutations at S65T and F64L making it 35-fold brighter in fluorescence relative to wild-type GFP. This variant was also found to be less prone to aggregation when compared to WT-GFP. EGFP has been optimised for excitation at 489 nm and emission at 508 nm. EGFP emits green fluorescence in its non-denatured state and has a yellow/green colour due to maximum absorbance at 489 nm, although the protein also has a broad absorbance spectrum between 400 nm and 480 nm.

Thus, GFP and variants, homologues and derivatives thereof are examples of suitable proteins for use in the present invention.

Similarly, the GFP structural homologue, wild-type DsRed (WT-DsRed) is a red fluorescent protein consisting of a beta-barrel structure, which also loses its characteristic red colour and fluorescence on denaturation. DsRed was isolated from the Discosoma species and has a tetrameric protein structure with each monomer having a molecular weight of 28 kDa. The chromophore activity (colour and fluorescence) which is intrinsic to DsRed is produced from an internal side chain cyclisation of the Gln-Tyr-Gly tripeptide (residues 66-68). DsRed and variants and derivatives thereof are examples of suitable proteins for use in the present invention. DsRed is known to be very stable, which is also an advantage in the present invention.

WT-DsRed exhibits its distinctive fluorescence and absorbance properties after undergoing a maturation process that takes several days. This slow maturation can be an undesirable property. A mutant form of WT-DsRed with mutations at R2A, K5E, K9T, V105A, I161T and S197A, known as DsRedExpress2 (DsRed2), was thus subsequently engineered for enhanced solubility and faster chromophore maturation; it was also shown to be non-cytotoxic in bacterial and mammalian cells upon expression, allowing it to be easily expressed in large quantities. In the present invention, the inventors have observed particularly good results using DsRed2, which has a red colour due to maximum absorbance at 559 nm (with a broad absorbance spectrum between 425 nm and 575 nm). On denaturation, the protein becomes colourless, thereby resulting in a decrease in intensity of absorbance at 559 nm.

Other non-limiting examples of suitable FPs include blue fluorescent protein (EBFP, EBFP2, Azurite, mKalama); cyan fluorescent protein (ECFP, Cerulean, CyPet); yellow fluorescent protein derivatives (YFP, Citrine, Venus, YPet); red fluorescent FPs such as monomeric TagRFP1, mKate2, dimeric TurboRFP, TurboFP635 and mCherry.

For use in the present invention, the FPs are generally modified to introduce or improve the binding of an accessory component. A preferred modification is polyhistidine-tagging and FPs so-modified are used along with imidazole or another polyhistidine-binding ligand as the accessory component.

In the case of DsRed, the protein is tetrameric so the addition of a single polyhistidine tag to each protein monomer will result in a protein complex with four polyhistidine tags. GFP is dimeric at higher concentrations (for example, 100 μM and above), so the addition of a single polyhistidine tag to each protein monomer will result in a protein complex with two polyhistidine tags under such conditions. More than one polyhistidine tag may be provided on any monomeric protein or other subunit.

The inventors have observed particularly good results using polyhistidine-tagged DsRed2, which in its native state has a red colour due to maximum absorbance at 559 nm, with a broad absorbance spectrum between 425 nm and 575 nm. On denaturation by denaturing ice crystals, the protein becomes colourless, thereby resulting in a decrease in intensity of absorbance at 559 nm. This change in state from the non-denatured to the denatured state may be further visualised by the loss of associated red fluorescence with excitation of the protein at 575 nm and emission at 583 nm.

Polyhistidine-tagged EGFP is also particularly suitable for use in the present invention. EGFP emits green fluorescence in its non-denatured state and has a yellow/green colour due to maximum absorbance at 489 nm, although the protein also has a broad absorbance spectrum between 400 nm and 480 nm. Denaturation of the protein by denaturing ice crystals results in the loss of absorbance at 489 nm and consequently change in the associated colour indication from green to colourless. This change in state from the non-denatured to the denatured state may be further visualised by the loss of green fluorescence with excitation of the protein at 489 nm and emission at 508 nm.

In another embodiment of the present invention, the protein may be modified to be coloured or fluorescent, for instance by modification with a suitable chromophore or fluorophore. Proteins may be made coloured or fluorescent by means of labelling via chemical modification or cross-coupling to other proteins or other molecules. Fluorescent labeling is the process of covalently attaching a fluorophore to another molecule, such as a protein or nucleic acid. This is generally accomplished using a reactive derivative of the fluorophore that selectively binds to a functional group contained in the target molecule. The most commonly labelled molecules are antibodies, proteins, amino acids and peptides which are then used as specific probes for detection of a particular target. Fluorescent labels are generally used for increasing the detection of a protein because fluorescence spectroscopy is a highly sensitive method of detection compared to absorbance based methods. Fluorescently labelled proteins may be used in fluorescence microscopy, flow cytometry or fluorescence instrumentation such as plate readers to measure fluorescent based assays (i.e. interaction of biomolecules). Fluorescence labelling of proteins also allows the sensitive detection in biochemical analysis of protein binding, western blot assays, and other immunoanalytical methods. Kits are readily available that contain the components needed to carry out fluorescent labelling reaction.

Reactive groups include isothiocyanate derivatives such as FITC and TRITC (derivatives of fluorescein and rhodamine) which are reactive towards primary amines to form a thioureido linkage between the compound of interest and the dye; succinimidyl esters such as NHS-fluorescein which are reactive towards amino groups to form an amido bond; and maleimide activated fluorophores such as fluorescein-5-maleimide which readily react with sulfhydryl groups. The sulfhydryl group adds to the double bond of the maleimide.

The reaction of a reactive dye with another molecule results in a stable covalent bond formed between the fluorophore and the labelled molecule. Following a fluorescent labelling reaction, it is often necessary to remove any non-reacted fluorophore from the labelled target molecule. This is often accomplished by size exclusion chromatography, taking advantage of the size difference between fluorophore and labelled protein. Fluorophores may interact with the separation matrix and reduce the efficiency of separation. For this reason, specialised dye removal columns that account for the hydrophobic properties of fluorescent dyes are sometimes used. Commonly used fluorescent dyes include, but are not restricted to, fluorescein, rhodamine, Alexa Fluors, Dylight fluors, ATTO Dyes and BODIPY.

Through protein engineering and expression it is possible to produce enzymes that produce a coloured, fluorescent or bioluminescent indication in the presence of corresponding enzyme substrates. For example, His6-tagged enzymes such as horse-radish peroxidise (HRP), luciferase and alkaline phosphatase may be expressed. These enzymes are commonly used for immunological based assays. Such enzymes are suitable proteins for use in the present invention. By way of example, the protein may comprise a non-denatured enzyme having the ability to convert its substrate into a visible coloured product, whereas denaturation of the enzyme by denaturing ice-crystals results in inactivation of the catalytic protein structure, thereby visibly changing or removing the colour indication. A further example is the use of a coloured substrate that is converted to a different colour or rendered colourless by an enzymatic reaction.

The protein for use in the present invention may be a fusion protein. Fusion proteins or chimeric proteins are proteins created through the joining of two or more genes which originally coded for separate proteins. Usually fusion proteins can be arranged as tandem constructs or placed at the N- or C-terminus of a protein. Translation of this ‘fusion gene’ results in a single recombinant polypeptide with functional properties derived from each of the original proteins

By way of example, the fusion protein may comprise at least one coloured or fluorescent protein (such as an FP) and an enzyme. Such a fusion protein can provide two simultaneous indications of denaturing ice crystal formation in the sample: a change in colour or fluorescence indications associated with the non-denatured and denatured states of the coloured or fluorescent protein and also a change in the indication resulting from an associated change of enzymatic interaction with a corresponding substrate.

In another example, the fusion protein comprises a bioluminescent enzyme and an FP. Bioluminescence is a naturally occurring form of chemiluminescence where energy is released by a chemical reaction in the form of light emission. The bioluminescent enzyme (e.g. luciferase) in the presence of its substrate (e.g. luciferin) partakes in bioluminescence energy transfer to its FP fusion partner (e.g. GFP). This results in green luminescence of the fusion product that can be observed in the dark.

The protein biosensor may be improved by the incorporation of one or more metal binding domains, such as the known LBP peptide sequences or other domains that are known to bind or chelate to metal ions. By way of example, the known LBP peptide sequence YIDTNNDGWYEGDELLA may be used, which binds to lanthanide ions (Su et al. Journal of the American Chemical Society 130.5 (2008): 1681-87). FPs engineered with binding sites or tags for metal ions are particularly sensitive to ice crystal denaturation. For example, it is known that proteins with a His-tag can bind Ni²⁺, Cu²⁺ and Co²⁺. Increasing the number of metal binding domains, for instance the LBP, a tag which binds lanthanide ions, results in a biosensor protein with increased denaturation sensitivity, giving a rapid rate of change in indication on ice-crystal denaturation.

The invention will now be further described with reference to the following non-limiting examples and accompanying figures in which:

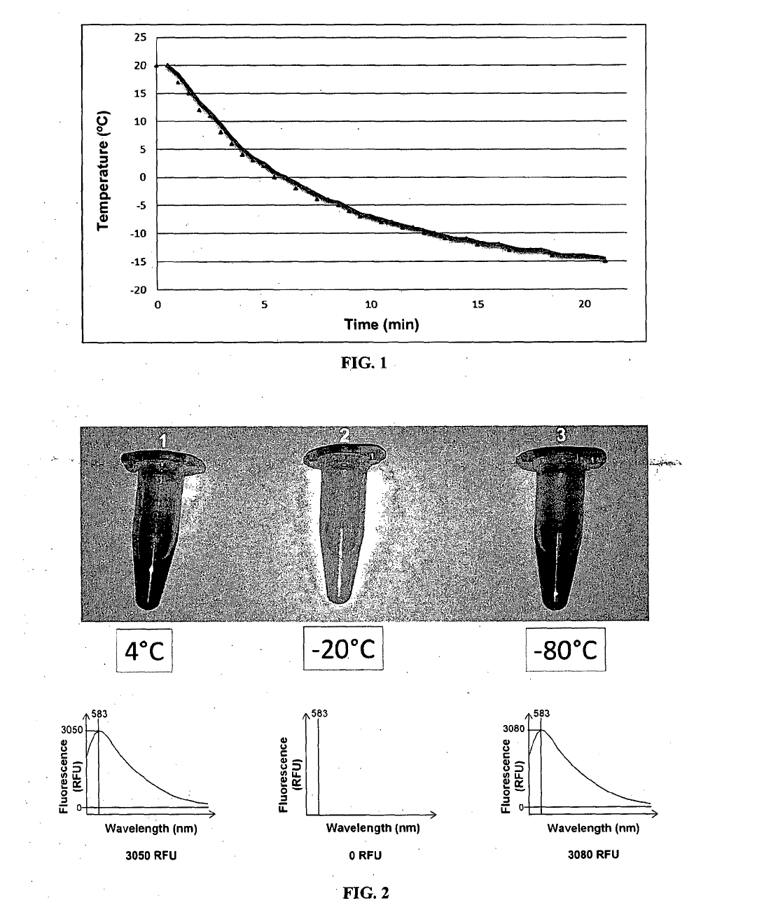

FIG. 1 shows the freezing rate of an aqueous phosphate buffered saline (PBS) sample in a domestic freezer at −20° C. The temperature of a 300 μl vial containing a buffer composition of 300 mM NaCl, 150 mM imidazole, 50 mM NaH₂PO₄, pH 8.0 was monitored with respect to time. A typical freezing rate of 4° C./min was observed.

FIG. 2 shows the colour and fluorescence indications of a representative biosensor according to the present invention in different test samples stored in tubes at 4° C. (Tube 1), −20° C. (Tube 2) and −80° C. (Tube 3), respectively. His-tagged DsRed2 was used at 2 mg/ml with 150 mM imidazole in PBS (i.e. 50 mM NaH₂PO₄, 300 mM NaCl, pH 8.0). After storage for 15 hours the biosensor in Tubes 1 and 3 remained red-coloured, whereas Tube 2 stored at −20° C. showed a loss in red colour and a complete loss in red fluorescence (excitation 559 nm emission 583 nm) as measured in relative fluorescence units (RFU).

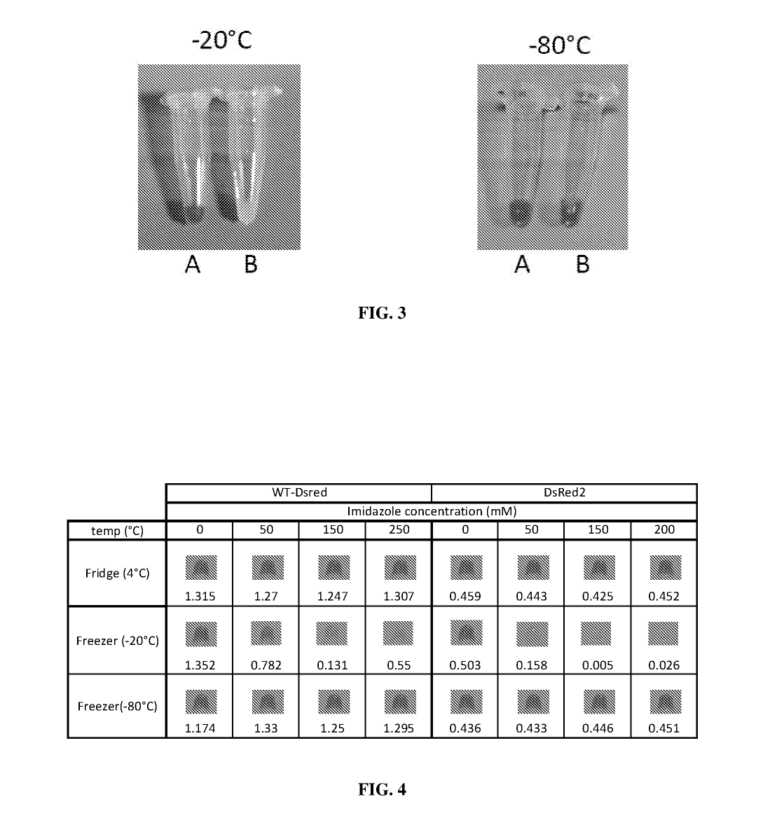

FIG. 3 shows the colour of (His6)-tagged DsRed2 samples at 2 mg/ml with 150 mM imidazole in PBS (i.e. 50 mM NaH₂PO₄, 300 mM NaCl, pH 8.0) frozen at −20° C. or −80° C. Samples ‘A’ were frozen for 10 min and samples ‘B’ were frozen overnight (15 hours). The samples are shown in the frozen state. These results clearly indicate that freezing the (His6)-tagged DsRed2 at either −20° C. or −80° C. for 10 mins had no effect on the colour observed (samples A). However, on freezing for a longer period of 15 hours the −20° C. sample was denatured and became colourless, owing to the formation of large crystals by the slow rate of freezing. By contrast, a (His6)-tagged DsRed2 sample frozen at −80° C. did not lose its red colour as the faster rate of freezing allowed the formation of smaller crystals which do not induce ice-crystal denaturation of the protein.

FIG. 4 shows the colour and absorbance of polyhistidine-tagged WT-DsRed (1.0 mg/ml) and polyhistidine-tagged DsRed2 (0.5 mg/ml) proteins stored for 15 hours at three different temperatures (4° C., −20° C. and −80° C.) with increasing concentrations of imidazole (i.e. 0, 50, 150 and 250 mM imidazole) in PBS. The figure shows individual photographs of each protein in an Eppendorf™ tube and its absorbance value at wavelength 559 nm. The results show the visual red-to-colourless cryogenic sensitivity at −20° C. Notably, DsRed2 showed better sensitivity by changing colour at 50 mM imidazole compared to WT-DsRed that had a similar response at 150 mM imidazole. 4° C.-refrigerated and the −80° C.-frozen DsRed proteins were unaffected and had similar absorbance values.

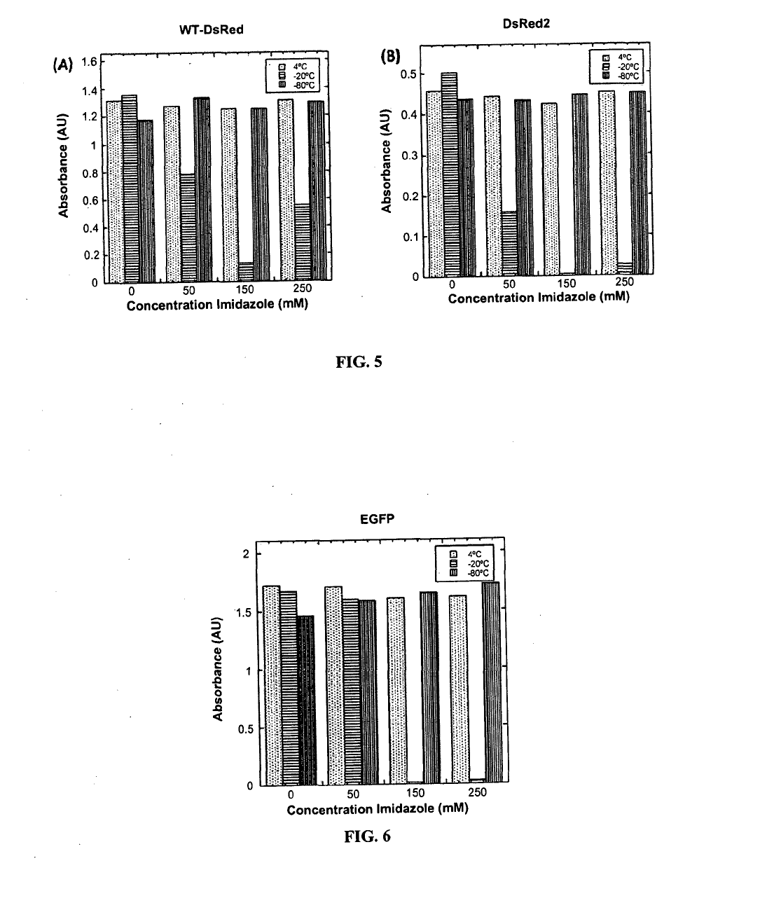

FIG. 5 shows the plotted results of FIG. 4, where the absorbance at 559 nm was measured using a spectrophotometer versus increasing imidazole concentrations (i.e. 0, 50, 150 and 250 mM imidazole) in PBS of (A) (His6)-tagged WT-DsRed at 1.0 mg/ml and (B) (His6)-tagged DsRed2 protein at 0.5 mg/ml stored at three different temperatures (4° C., −20° C. and −80° C.) for 15 hours. In general, the addition of increasing concentrations of imidazole resulted in the reduction in absorbance only when the samples were frozen at −20° C., with optimal reduction observed with 150 mM Imidazole.

FIG. 6 shows the absorbance at 489 nm versus increasing imidazole concentrations in PBS with polyhistidine-tagged EGFP at 1 mg/ml in PBS after storage at three different temperatures (4° C., −20° C. and −80° C.) for 15 hours. A reduction in absorbance was observed when the samples were frozen at −20° C., with optimal reduction observed with 150 mM and 250 mM imidazole.

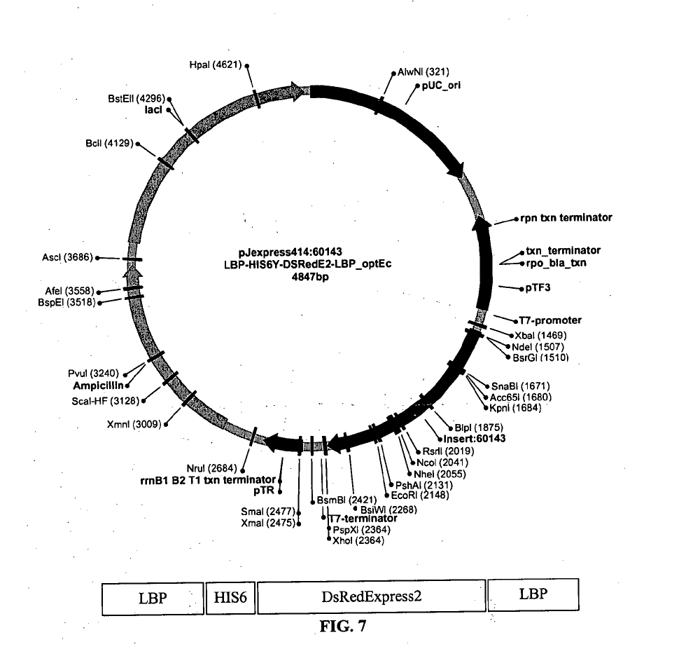

FIG. 7 shows a schematic representation of a plasmid used for expression of (His6)-tagged DsRed2 in E. coli and a scheme showing a fusion protein comprising DsRed2 and lanthanide binding peptide (LBP) and His6-tag sequences. This construct expressed in the plasmid gave very high levels of expression (around 20 mg/1) compared to WT-DsRed (around 5 mg/1) expressed using the plasmid pRSETA (Invitrogen, USA).

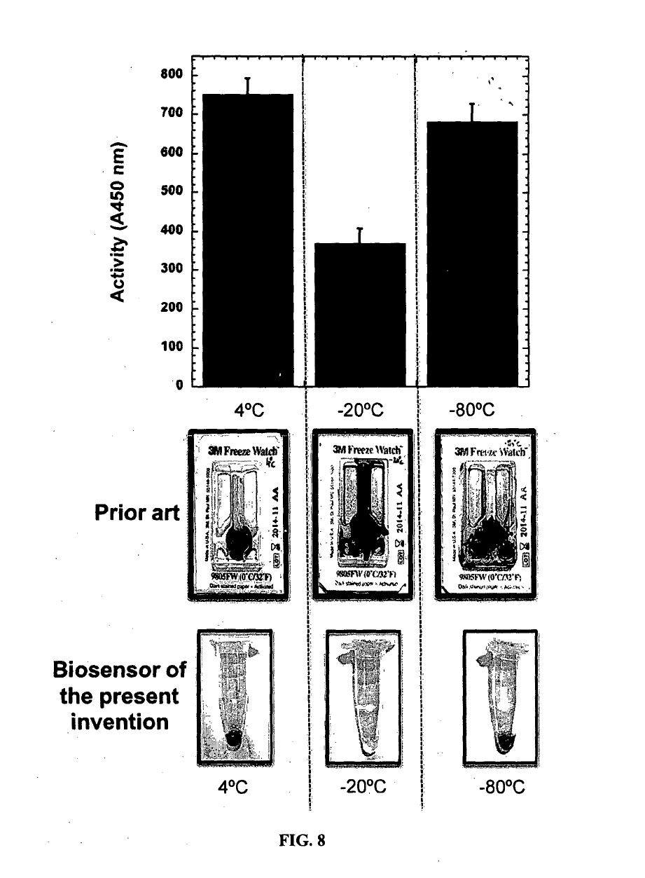

FIG. 8 shows the effects of storage at 4° C., −20° C. and −80° C. on the activity of the enzyme horse radish peroxidase (HRP) measured at absorbance 450 nm, 3M Freeze Watch® devices and a representative biosensor according to the present invention (his-tagged DsRed2 at 2 mg/ml with 150 mM imidazole in PBS). Storage at −20° C. resulted in a loss of around 50% HRP activity, presumably through ice-crystal denaturation of the enzyme, but freezing at −80° C. had little effect. This correlated directly with the colour indications provided by the biosensor of the present invention, which was colourless after storage at −20° C. but remained red at −80° C. The 3M Freeze Watch® device registered a freezing event at both −20° C. and −80° C. and did not distinguish between the two conditions.

### EXAMPLES

### Fluorescent Protein Constructs

Enhanced green fluorescent protein (EGFP), a mutated version of GFP, was engineered with an N-terminal (His6)-tag and thrombin cleavage site. The GFP structural homologue wild-type DsRed (WT-DsRed) was engineered with an N-terminal (His6)-tag. DsRedExpress2 (DsRed2), a mutated version of WT-DsRed, was engineered with an N-terminal (His6)-tag and a lanthanide binding peptide (LBP) tag at both the N-terminus and the C terminus.

### Fluorescent Protein Expression and Purification

Plasmids containing the DNA sequences of either (His6)-tagged DsRed, (His6)-tagged DsRed2 (see FIG. 8), (His6)-tagged EGFP were transformed into chemically competent E. coli BL21(DE3) cells. 5 ml of an overnight culture grown at 37° C. in Luria Bertani (LB) medium (10 g Bacto tryptone, 5 g yeast extract, 10 g NaCl per litre) was then inoculated into 500 ml LB medium containing 100 μg/ml ampicillin. The cultures were grown at 37° C. with shaking at 200 rpm to OD₆₀₀=0.8. Isopropyl-β,D-thiogalactopyranoside (IPTG) was added to a final concentration of 1 mM and the cells were grown overnight at 37° C. for WTDsRed and DsRed2 and 25° C. for EGFP with shaking at 200 rpm, and then collected by centrifugation at 3,500 rpm for 30 min at using a Beckmann Coulter model centrifuge. After centrifugation the cell pellets were resuspended in 50 ml PBS buffer (50 mM NaH₂PO₄, 300 mM NaCl, pH 8.0) consisting of 20 mM imidazole, sonicated using a SoniPrep 150 (MSE, UK) and centrifuged at 7,000 rpm using a Heraeus Biofuse Statos centrifuge (Jencons-PLS, UK). The cell supernatant was loaded onto a Ni-NTA column for purification (metal affinity chromatography). DsRed, DsRed2 and EGFP samples were eluted from the Ni-NTA column using elution buffer (250 mM imidazole, 50 mM NaH₂PO₄, 300 mM NaCl, pH 8.0). All proteins were dialyzed overnight against PBS (50 mM NaH₂PO₄, 300 mM NaCl, pH 8.0).

The protein absorbances for DsRed and DsRed2 were measured at 559 nm, and EGFP at 489 nm using a Nanodrop ND1000 (Thermo Fisher Scientific, USA) and the fluorescence measured using a NanoDrop ND3300 (Thermo Fisher Scientific, USA).

### Fluorescent Proteins as Indicators of Denaturing Ice-Crystal Formation

A series of freezing studies were undertaken at a range of different temperature conditions: a refrigerator at 4° C., a freezer at −20° C. and a freezer at −80° C.

The 4° C. fridge and −20° C. freezer was the ProfiLine model, from Liebherr (Liebherr-Great Britain Ltd, UK). As seen in FIG. 1, a typical freezing rate of 4° C./min was observed in the −20° C. freezer.

The −80° C. freezer was the Revco Model ULT1386-3-V39 (Kendro Laboratory products, USA).

Imidazole was used as an accessory component. The inventors studied the effects of storage at 4° C., −20° C. and −80° C. of (His6)-tagged WT-DsRed (1.5 mg/ml) or (His6)-tagged DsRed2 (1.25 mg/ml) in PBS in the presence of increasing amounts of imidazole. The DsRed2 protein was also tagged with a lanthanide-binding peptide (LBP) at the N-terminus followed by a His6-tag and another LBP tag at the C terminus (see FIG. 7). Absorbance was measured at wavelength 559 nm. As seen in FIGS. 4 and 5, storage of these modified fluorescent proteins at 4° C. had no effect on the visual and absorbance indications of either protein. Similarly, freezing at −80° C. had no effect on the visual and absorbance indications of the fluorescent proteins, indicating that there was no formation of denaturing ice crystals. In contrast, storage of the proteins at −20° C. in the presence of imidazole resulted in detectable changes in the visual and absorbance indications, thus indicating denaturation of the proteins by denaturing ice-crystal formation in the aqueous samples. The (His6)-tagged DsRed2 was found to have better cryogenic sensitivity than the (His6)-tagged WT-DsRed; the (His6)-tagged DsRed2 showed a clear change in visual indication from red to colourless at 50 mM imidazole whereas the (His6)-tagged WT-DsRed showed a similar change in colour indication at 150 mM imidazole (although for both proteins, the best results were observed at an imidazole concentration of 150 mM).

The insertion of the LBP tags in the DsRed2 variant created an enhanced cryogenic sensitivity (i.e. a colour change at a lower imidazole concentration), compared to WT-DsRed as shown in FIG. 4 and FIG. 5.

FIG. 2 shows the visual indications and fluorescence spectra associated with samples of (His6)-tagged DsRed2 (2 mg/ml) in the presence of 150 mM imidazole after storage at 4° C., −20° C. and −80° C. The visual change from red to colourless after storage at −20° C., and hence formation of denaturing ice crystals, is readily apparent and there was a complete loss of red fluorescence indication under these conditions. On the other hand, no changes in colour or fluorescence indications were observed after storage at 4° C. or −80° C., indicating that the fluorescent protein remained in the non-denatured state.

The samples shown in FIG. 2 were subjected to repeated cold storage and intervening storage at room temperature, in order to investigate the effects of multiple freeze-thaw cycles on the visual indications provided by the (His6)-tagged DsRed2. The results are shown in Table 1 below.

TABLE 1

| | Initial state/ | State/colour | | |
|---|---|---|---|---|
| | colour | overnight | State/ | Samples re-frozen |
| | (before | (after | colour on | and re-thawed |
| Temperature | freezing) | freezing) | thawing | (3 months) |
| 4° C. | Liquid/ | Liquid/ | Liquid/ | Liquid/ |
| fridge | red | red | red | red |
| −20° C. | Liquid/ | Frozen/ | Liquid/ | Liquid/ |
| freezer | red | clear | clear | clear |
| −80° C. | Liquid/ | Frozen/ | Liquid/ | Liquid/ |
| freezer | red | red | red | red |

Repeated storage at 4° C. had no effect on the colour indication, the sample remaining red throughout. Similarly, storage at −80° C., thawing at room temperature and repeated re-freezing and re-thawing had no effect on the colour indication, the sample remaining red even after 3 months. In contrast, storage at −20° C. resulted in a change of colour indication from red to colourless. Advantageously, this was readily detectable in the frozen state, so there was no need to thaw the sample in order to detect the indication of denaturation. The sample remained colourless even after thawing and further −20° C. freeze-thaw cycles. The indication of denaturation was therefore detectable even after the conditions were changed so that the formation of denaturing ice crystals was removed.

These results are confirmed in FIG. 3, which shows the (His6)-tagged DsRed2 samples in the frozen state. Freezing for 10 mins at −20° C. or −80° C. (samples ‘A’) had no effect, in other words there was no denaturing ice-crystal formation. Storage at −20° C. for 15 hours resulted in a clearly observable visual change from red to colourless. This was seen in the frozen state. Storage at −80° C. did not have an effect on the colour indication.

These effects were confirmed using (His6)-tagged EGFP in the presence of increasing concentrations of imidazole (i.e. 0, 50, 150 and 250 mM imidazole) in the presence of PBS (50 mM NaPO₄, 300 mM NaCl, pH pH8.0). A detectable change of visual indication from green to colourless was observed after storage at −20° C., whereas storage at 4° C. or −80° C. had no effect. Optimal results were observed at 150 mM and 250 mM imidazole (see FIG. 6).

FIG. 8 shows the effects of storage at 4° C., −20° C. and −80° C. on the activity of the enzyme horse radish peroxidase (HRP) measured at absorbance 450 nm, 3M Freeze Watch® devices and a representative biosensor according to the present invention (his-tagged DsRed2 at 2 mg/ml with 150 mM imidazole in PBS). The HRP assay was performed with each sample at 25° C., in a total assay volume of 1500 in a 96-well microtitre plate. This consisted of 500 HRP (0.625 μg/ml) with subsequent addition of 500 of TMB substrate reagent (3, 3′, 5, 5′ tetramethyl benzidine at 0.1 mg/ml, 100 mM citrate acetate buffer pH 5.6 and 0.05% H₂O₂) which was then mixed by pipetting. The microplate was incubated for 5 min at 25° C., 500 of stop solution (0.25M HCl) was added and the absorbance was read at 450 nm (BMG POLARstar, BMG-Labtech, Germany). The HRP results clearly demonstrate that at −20° C. there is a loss of approximately 50% of HRP activity, presumably through ice-crystal denaturation. The biosensor composition of the present invention has provided an indication of this ice-crystal denaturation event at −20° C. by a red to colourless change yet there is no significant change of colour at −80° C. (i.e. no ice-crystal denaturation). Notably the colour change of the biosensor of the present invention correlates directly to activity of HRP whereas the Freeze Watch® device has only registered a physical freezing event (the glass is ruptured and a black ink is released in the vial). Therefore, in contrast to the 3M Freeze Watch® device, the biosensor of the present invention not only indicates a freezing event but also indicates ice-crystal damage to proteins. Thus, the present invention provides a means for detection and indication of denaturing ice-crystal formation within the contents of a packaged product, whereas the 3M Freeze Watch® device indicates only whether the package itself has been frozen.

1. The use of a protein as a biosensor to indicate denaturing ice-crystal formation in a sample, wherein the protein is denaturable by denaturing ice crystals from a non-denatured state to a denatured state and wherein said non-denatured and denatured states are associated with detectably different indications, said protein comprising a ligand-binding domain.

2. The use according to claim 1, wherein the protein is at least partially irreversibly denaturable by denaturing ice crystals.

3. (canceled)

4. (canceled)

5. The use according to claim 1, wherein the non-denatured and denatured states of the protein are associated with detectably different colour indications.

6. The use according to a claim 1, wherein the protein is a fluorescent protein.

7. The use according to claim 6, wherein the protein comprises a protein selected from: DsRed, DsRed2, EGFP, GFP, ECFP, Cerulean, CyPet, YFP, Citrine, Venus, YPet, TagRFP1, mKate2, TurboRFP, TurboFP635 and mCherry, and analogues and derivatives thereof.

8. The use according to claim 7, wherein the protein comprises DsRed2.

9. (canceled)

10. The use according to claim 1, wherein the protein is a fusion protein.

11. (canceled)

12. (canceled)

13. The use according to claim 1, wherein the ligand-binding domain and its corresponding ligand are selected from: polyhistidine and imidazole, glutathione S-transferase (GST) and gluthathione, streptavidin and biotin, luciferase and luciferin, and analogues and derivatives thereof.

14. The use according to claim 13, wherein the ligand and ligand-binding domain comprise imidazole, or an analogue, derivative or functional equivalent thereof, and polyhistidine, respectively.

15. The use according to claim 14, wherein the concentration of ligand is 10-500 mM, 25-400 mM, 50-300 mM, 75-250 mM, 100-200 mM, 125-175 mM or around 150 mM; and the concentration of the polyhistidine-tagged protein is 0.1-5 mg/ml, 0.25-4 mg/ml, 0.5-3 mg/ml, 0.75-2 mg/ml, 1-1.5 mg/ml or around 1 mg/ml.

16. The use according to claim 1, wherein the protein is provided with at least one metal binding domain.

17. The use according to claim 16, wherein the metal binding domain comprises the peptide sequence YIDTNNDGWYEGDELLA (SEQ ID NO: 1).

18. The use according to claim 1, wherein the sample is selected from one or more of the following: pharmaceuticals, antibodies, enzymes, biopharmaceuticals, laboratory reagents, tissues, cells, cell research products, blood, blood products and food products.

19. A biosensor composition for indicating denaturing ice-crystal formation in a sample, the composition comprising a protein, wherein the protein is denaturable by denaturing ice crystals from a non-denatured state to a denatured state and wherein said non-denatured and denatured states are associated with detectably different indications, said protein comprising a ligand-binding domain.

20. (canceled)

21. (canceled)

22. A biosensor composition according to claim 19, wherein the biosensor composition further comprises
a ligand bindable by the ligand-binding domain.

23. A biosensor composition according to claim 19, wherein the biosensor composition further comprises water.

24. A biosensor composition according to claim 23, wherein the biosensor composition further comprises one or more of the following components: PBS, NTA, NTA derivatives, 3-{[tris(hydroxylmethyl)methyl]amino}propanesulfonic acid, N,N-bis-(2-hydroxyethyl)glycine, tris(hydroxymethyl)methylamine, N-tris-(hydroxyl-methyl)methylglycine, 4-2-hydroxyethyl-1-piperazineethanesulfonic acid, 3-(N-morpholino)propanesulfonic acid, piperazine-N,N′-bis(2-ethanesulfonic acid), saline sodium citrate, 2-(N-morpholino)ethanesulfonic acid, acetic acid, sodium acetate, K₂HPO₄, KH₂PO₄, KCl and EDTA.

25. A biosensor composition according to claim 19, wherein the protein is at least partially irreversibly denaturable by denaturing ice crystals.

26. (canceled)

27. (canceled)

28. A biosensor composition according to claim 19, wherein the non-denatured and denatured states of the protein are associated with detectably different colour indications.

29. A biosensor composition according to claim 19, wherein the protein is a fluorescent protein.

30. A biosensor composition according to claim 29, wherein the protein comprises a protein selected from: DsRed, DsRed2, EGFP, GFP, ECFP, Cerulean, CyPet, YFP, Citrine, Venus, YPet, TagRFP1, mKate2, TurboRFP, TurboFP635 and mCherry, and analogues and derivatives thereof.

31. A biosensor composition according to claim 30, wherein the protein comprises DsRed2.

32. (canceled)

33. A biosensor composition according to claim 19, wherein the protein is a fusion protein.

34. A biosensor composition according to claim 22, wherein the ligand and ligand-binding domain are selected from: imidazole and polyhistidine, glutathione and glutathione S-transferase (GST), biotin and streptavidin, luciferin and luciferase, and analogues and derivatives thereof.

35. A biosensor composition according to claim 34, wherein the ligand and ligand-binding domain comprise imidazole, or an analogue, derivative or functional equivalent thereof, and polyhistidine, respectively.

36. A biosensor composition according to claim 35, wherein the concentration of ligand is 10-500 mM, 25-400 mM, 50-300 mM, 75-250 mM, 100-200 mM, 125-175 mM or around 150 mM; and the concentration of the polyhistidine-tagged protein is 0.1-5 mg/ml, 0.25-4 mg/ml, 0.5-3 mg/ml, 0.75-2 mg/ml, 1-1.5 mg/ml or around 1 mg/ml.

37. A biosensor composition according to claim 19, wherein the protein is provided with at least one metal binding domain.

38. A biosensor composition according to claim 37, wherein the metal binding domain comprises the peptide sequence YIDTNNDGWYEGDELLA (SEQ ID NO: 1).

39. A biosensor composition according to claim 19, wherein the sample is selected from one or more of the following: pharmaceuticals, antibodies, enzymes, biopharmaceuticals, laboratory reagents, tissues, cells, cell research products, blood, blood products and food products.

40. A method of indicating denaturing ice-crystal formation in a sample stored under storage conditions, the method comprising:
(i) storing under said storage conditions a biosensor composition comprising a protein, wherein the protein is denaturable by denaturing ice crystals from a non-denatured state to a denatured state and wherein said non-denatured and denatured states are associated with detectably different indications and wherein said protein comprises a ligand-binding domain; and water; and
(ii) detecting any change of indication.

41. A method according to claim 40, wherein the protein is at least partially irreversibly denaturable by denaturing ice crystals.

42. (canceled)

43. (canceled)

44. A method according to claim 40, wherein the non-denatured and denatured states of the protein are associated with detectably different colour indications.

45. A method according to claim 40, wherein the protein is a fluorescent protein.

46. A method according to claim 45, wherein the protein comprises a protein selected from: DsRed, DsRed2, EGFP, GFP, ECFP, Cerulean, CyPet, YFP, Citrine, Venus, YPet, TagRFP1, mKate2, TurboRFP, TurboFP635 and mCherry, and analogues and derivatives thereof.

47. A method according to claim 46, wherein the protein comprises DsRed2.

48. (canceled)

49. A method according to claim 40, wherein the protein is a fusion protein.

50. (canceled)

51. (canceled)

52. A method according to claim 40, wherein the accessory component is a ligand and the protein is provided with at east one corresponding ligand binding domain selected from: polyhistidine and imidazole, glutathione S-transferase (GST) and gluthathione, streptavidin and biotin, luciferase and luciferin, and analogues and derivatives thereof.

53. A method according to claim 52, wherein the ligand and ligand-binding domain comprise imidazole, or an analogue, derivative or functional equivalent thereof, and polyhistidine, respectively.

54. A method according to claim 53, wherein the concentration of ligand is 10-500 mM, 25-400 mM, 50-300 mM, 75-250 mM, 100-200 mM, 125-175 mM or around 150 mM; and the concentration of the polyhistidine-tagged protein is 0.1-5 mg/ml, 0.25-4 mg/ml, 0.5-3 mg/ml, 0.75-2 mg/ml, 1-1.5 mg/ml or around 1 mg/ml.

55. A method according to claim 40, wherein the protein is provided with at least one metal binding domain.

56. A method according to claim 55, wherein the metal binding domain comprises the peptide sequence YIDTNNDGWYEGDELLA (SEQ ID NO: 1).

57. A method according to claim 40, wherein the sample is selected from one or more of the following: pharmaceuticals, antibodies, enzymes, biopharmaceuticals, laboratory reagents, tissues, cells, cell research products, blood, blood products and food products.

58. A method according to claim 40, wherein the biosensor composition further comprises one or more of the following components: PBS, NTA, NTA derivatives, 3-{[tris(hydroxylmethyl)methyl]amino}propanesulfonic acid, N,N-bis-(2-hydroxyethyl)glycine, tris(hydroxymethyl)methylamine, N-tris-(hydroxyl-methyl)methylglycine, 4-2-hydroxyethyl-1-piperazineethanesulfonic acid, 3-(N-morpholino)propanesulfonic acid, piperazine-N,N′-bis(2-ethanesulfonic acid), saline sodium citrate, 2-(N-morpholino)ethanesulfonic acid, acetic acid, sodium acetate, K₂HPO₄, KH₂PO₄, KCl and EDTA.

59. A method according to claim 40, wherein the method further comprises a step of exposing the protein to UV light.

60. A method according to claim 40, wherein the biosensor composition is introduced into or otherwise combined with the sample prior to storage.

61. A method according to claim 40, wherein the biosensor composition is stored separately from but in conjunction with the sample.

62. A method according to claim 61, wherein the biosensor composition is stored separately in or on containment means containing the sample.

63. A method according to claim 62, wherein the biosensor composition is applied to packaging of the sample.

64. (canceled)

65. A method according to claim 62, wherein the biosensor composition forms part of a bar code or other identification means.

66. (canceled)

67. A method according to claim 40, wherein the biosensor composition further comprises a ligand bindable by the ligand-binding domain.


SEQUENCE LISTING (1 sequence)

SEQ ID NO: 1 - protein, 17 aa, Artificial Sequence
  Synthesized
YIDTNNDGWYEGDELLA